(19) United States
(12) Patent Application Publication
(10) Pub. No.: US 2023/0363765 A1
(43) Pub. Date: Nov. 16, 2023

(54) Ring of Life

(71) Applicant: John Arthur Sievert, Chula Vista, CA (US)

(72) Inventor: John Arthur Sievert, Chula Vista, CA (US)

(21) Appl. No.: 17/741,904

(22) Filed: May 11, 2022

(51) Int. Cl. A61B 17/132

(52) U.S. Cl. CPC A61B 17/1325

(57) ABSTRACT

The Ring of Life tourniquet system is a motorized tourniquet system with intelligent pads. This invention uses a metal band and pads to apply radial pressure to stop bleeding from an affected. The invention uses built-in injection ports that enable an individual to administer antibiotic prophylaxis to traumatized tissue and other medications. This system is developed to prevent infection to an affected area for an extended period, track and record heart rate, uses GPS tracking for rapid response and emergency communications. The device and medication can be applied with one hand alone or with no professional medical treatment.

### FIELD OF THE INVENTION

The present invention relates to a novel flow restriction device, particularly a novel tourniquet device for restricting blood flow.

### BACKGROUND

This embodiment relates to a tourniquet system that stops arterial bleeding by blocking blood flow to the affected area. Tourniquets are used in the medical field, military forces, and consumer medical kits to prevent loss of life from blood loss. The tourniquets system can be operated using one hand when an individual is alone or without medical assistance.

Tourniquets prevent the loss of life in accidental, emergency, combat, and other related reasons where professional medical treatment is not available; an individual can quickly apply the tourniquet around the arm or the leg to prevent blood loss. Tourniquets can typically be applied using one hand. The tourniquet uses a metal band and pads to create radial pressure to the affected area preventing blood loss. Tourniquets applied around legs or arms are tightened to avoid blood loss, cause friction burns nerve damage, and do not treat an infection. A tourniquet that applies uneven pressure to the affected area can create other problems, including blood loss, reapplication of the tourniquet, unnecessary damage to muscle tissue. Individuals restricting blood flow during sustained injuries where medical professionals cannot reach or treat for long periods become critical in maintaining life. The extended periods where treatment is vital in preventing infections can cause further damage to the affected site where dirt, chemicals, or other foreign contaminants can enter the body from the compromised area. These contaminants can cause loss of limb, removal of infected tissue, or even death. Tourniquets need to be weather-resistant, easily accessible, durable, lightweight and treat the affected area with medications to sustain wellness. Most potential users tend not to carry devices unless they meet these requirements.

Accordingly, there is a need for an emergency lightweight tourniquet that can provide improved radial pressure to the wounded limb, restrict blood flow, treat infection, and prevent infection to the affected area. There is a further need for such a tourniquet that helps medical responders locate and communicate with individuals in areas or situations that complicate finding their position.

### BRIEF DESCRIPTION OF THE DRAWINGS

All fully assembled images have the top pads removed to demonstrate the position of the metal ring and internal device parts.

FIG. 1. It is an Isometric view with hidden lines shown

FIG. 2. It is an Isometric view of the invention fully assembled

FIG. 3. It is a bottom-up right-side Isometric view of the upper ring

FIG. 4. It is a top isometric view of the top upper ring

FIG. 5. Is a rear sectional view of the top right sectional

FIG. 6. Is a top sectional view of top right sectional

FIG. 7. Is a Front view upper ring

FIG. 8. Is a Front view upper ring

FIG. 9. Is a front sectional view of rear left sectional

FIG. 10. Is a back isometric view of the left rear sectional

FIG. 11. Is an Isometric view of the pad

FIG. 12. Is a back view of the pad

FIG. 13. It is an isometric view of electric motor control

FIG. 14. Is a Bottom view of electric motor control

### PARTS LIST

1 Lower ring right sectional
2 Lower ring left sectional
3 Metal band
4 Electrical motor control
5 Upper ring right sectional
6 Upper ring left Sectional
7 Bolt locking compartment
8 Injection port
9 Pad
10 Node holes
11 Electrical motor control slot
12 Electric motor control attachment slot
13 Bolt holes
14 Pad nodes
15 Start button
16 Tightening screw

The drawings are not necessarily to scale and may, in part, include exaggerated dimensions for clarity.

### SUMMARY OF THE INVENTION

The invention and other needs are addressed by the various embodiments and configurations of the present invention. The present invention comprises two upper sectional parts that make up the upper ring and two lower sectional parts that make up the lower ring. The upper ring includes injection ports, handle movement slot, bolt holes, and bolt locking compartment. The lower ring comprises a plastic bag removal slot, bolt locking compartment, and bolt holes.

In accordance with the embodiments of the present invention, the circular tourniquet ring comprises two injection ports used to administer medication using medication vials. The medications prevent infection or further infection of the compromised limb over an extended period.

In accordance with the embodiments of the present invention, the medical cover removal slot allows a cover to be placed over the compromised limb to prevent dirt, debris, chemicals, or other foreign material from coming in contact with the affected limb.

In accordance with the embodiments of the present invention, the bolt holes allow a bolt to pass through the upper and lower ring to increase the diameter of the tourniquet. The locking bolt compartment prevents the ring from the tourniquet returning to the original diameter.

In accordance with the embodiments of the present invention, the electric motor control and tightening screw controls the diameter of the metal ring. The electrical motor control is used to tighten the metal ring and press the pads around the limb to stop blood flow.

In accordance with the embodiments of the present invention, the pads have nodes that are used to guide the pads to the affected limb without contact from the metal ring. The pads prevent friction burns and nerve and muscle damage to the affected limb.

In accordance with the embodiments of the present invention, the metal ring’s circumference is determined by the electrical motor control and bolt locking compartment. Increases circumference to be placed on a compromised site where the metal band can be tightened, pushing padding to the affected limb to prevent blood loss.

In accordance with the embodiments of the present invention, the padding hole slots are used to keep the padding stationary till the metal band decreases circumference. The padding nodes come out of the padding hole slots and continue with the metal ring to perform radial pressure on the affected limb.

Various embodiments of the present invention are outlined in the attached figures. The claims provided a detailed description of the invention and embodied by the invention. However, it should be understood that this Summary of the Invention may not contain all aspects and embodiments of the present invention. The detailed description is not meant to be limiting or restrictive. The invention as disclosed herein is and will be understood by those of ordinary skill in the art to encompass apparent improvements and modifications to that.

Additional advantages of the present invention will become readily apparent from the following discussion, particularly when taken together with the accompanying drawings.

### DETAILED DESCRIPTION OF THE INVENTION

The present invention comprises a tourniquet that can be used with one hand by pressing the start button 15 engaging an electrical motor control 4 that reduces the metal band 3 radial diameter, adding pressure to the pads 9 applying pressure to stop blood flow on a limb. The device has an internal GPS used in emergencies and allows emergency communications once the start button 15 is pressed. In addition, the tourniquet provides improved bleeding stoppage by way of an inner metal band 3 tightening positioned within the center of the invention.

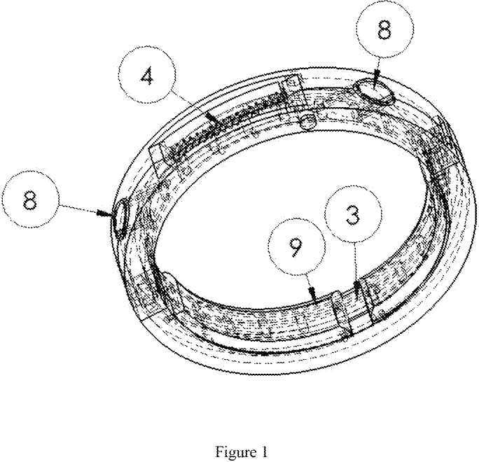

Referring to FIG. 1, a tourniquet bottom-up isometric view with hidden lines. The image shows a circular tourniquet with an electrical motor control 4 secured in the electric motor control attachment slot 12. The electrical motor control 4 manipulates a metal band 3 on the inner surface of the invention to force the band to reduce in size. The metal band 3 within the member’s center applies radial pressure and pushes padding 9 against the affected limb stopping blood flow. The bolt locking compartments 7 and bolt holes 13 locks the upper and lower rings into position to fit around an individual’s limb.

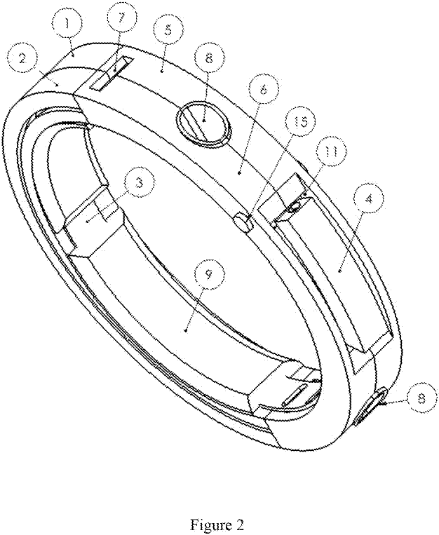

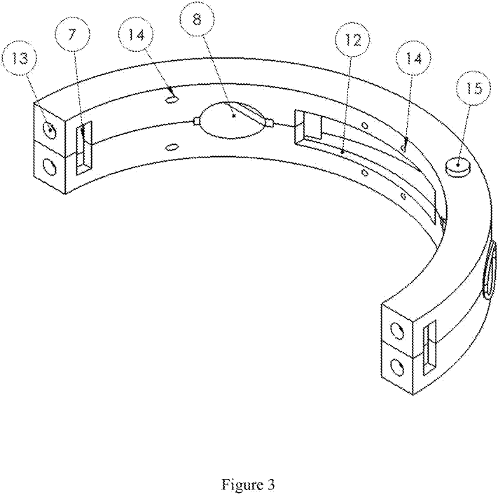

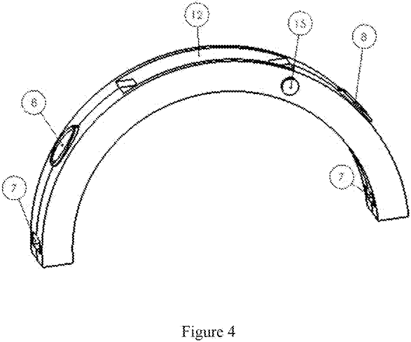

Referring to FIG. 2, a front view with no hidden lines is shown. The lower ring right sectional 1 is connected to the lower ring left sectional 2. The lower ring right sectional 1 has a medical cover removal slot 11 that applies a medical cover that will prevent chemicals, dirt or other substances from contacting the affected area.

Referring to FIG. 2, a top isometric view without hidden lines, the image shows an upper right 5 and left sectional 6 snapped together and forms two injection ports 8 where medications can be administered.

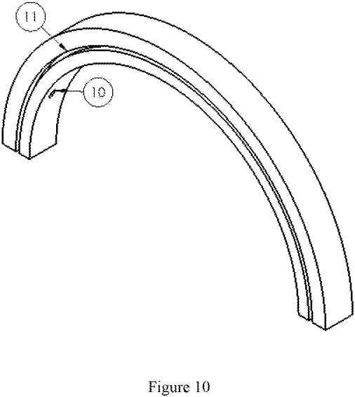

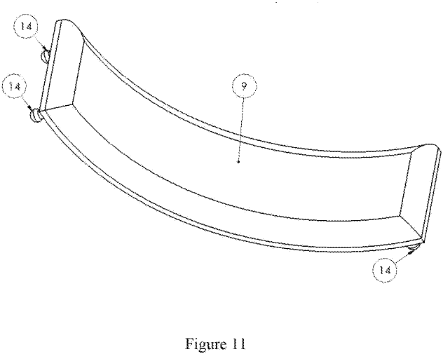

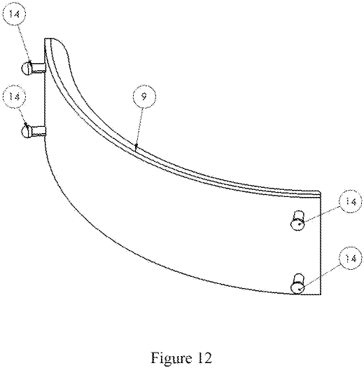

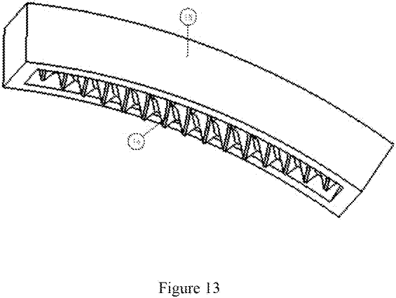

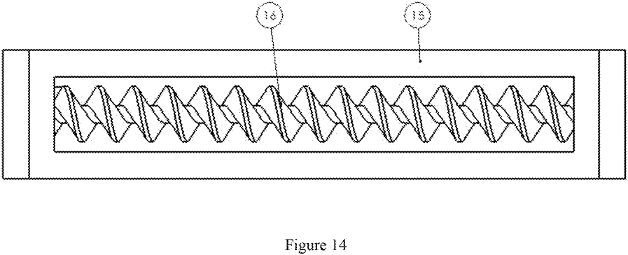

Referring to FIGS. 9 and 10, a front sectional view back sectional view without hidden lines. The lower ring right sectional 1 has a medical cover removal slot 11 that applies a medical cover that will prevent chemicals, dirt or other substances from contacting the affected area.

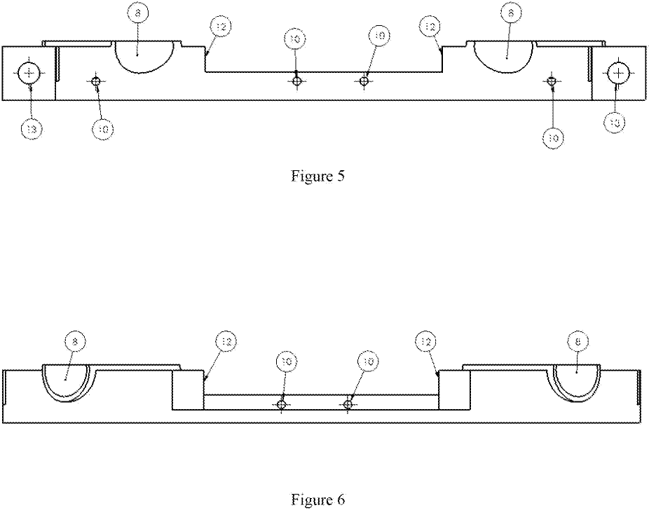

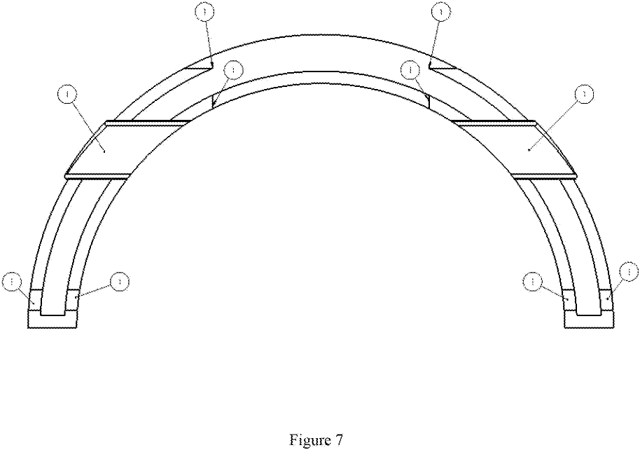

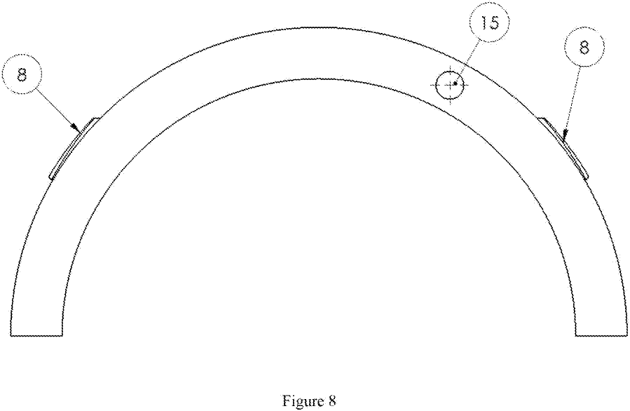

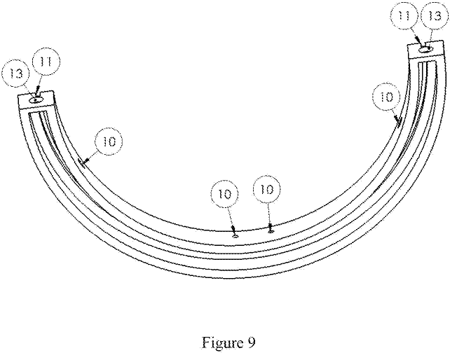

Referring to FIGS. 5 and 6, a sectional bottom view of the top ring and top sectional top view of the top ring without hidden lines are shown. The images show node holes 10 that are used in conjunction with padding 9. The pad nodes 14 fit inside the node holes 10 and help guide the padding 9 to the affected limb. The bolt hole 13 keeps the lower and upper rings connected using a bolt. The six members of the right 5 and left sectional 6 snap together and form the injection ports 8 and hand crack slot 12.

What is claimed:

1. A tourniquet for restricting the flow of blood in a limb, the tourniquet comprising:
(a) Two upper sectionals embodiments, right and left, that make up the upper ring where the two pieces combined form two injection ports, two-bolt locking compartments, and contains four bolt holes on the lower base of the upper ring.
(b) Two lower sectionals, right and left, make up the lower ring that forms two-bolt locking compartments to prevent bolt movement and four-bolt holes that house the bolts to attach to the upper ring.
(c) Sixteen node hole slots are used to steady the padding for radial pressure to be equal at all points on the residual limb.
(d) An electrical motor slot that attaches the electric motor control.

2. The tourniquet, as claimed in claim 1, is further comprised of four pads for radial compression to the affected limb.

3. The tourniquet, as claimed in claim 1, is further comprised of a metal band that increases and decreases in circumference using an electrical motor control. The metal band moves four pads using radial pressure to prevent blood loss from the affected limb.

4. The tourniquet, as claimed in claim 1, is further comprised of electric motor control with a tightening screw that reduces the circumference of a metal ring to apply radial pressure to the affected area.

5. The tourniquet, as claimed in claim 1, is further comprised of pad nodes that are used in conjunction with the node holes to prevent the padding from moving during radial compression.

6. The tourniquet, as claimed in claim 1, is further comprised of a medical cover slot that covers the affected limb to prevent further infection.

7. The tourniquet, as claimed in claim 1, comprises four bolt holes located and the base of the lower and upper ring.

8. The tourniquet, as claimed in claim 1, is further comprised of upper and lower sectional pieces that are snapped together to prevent embodiments from separating during radial compression.

9. The tourniquet, as claimed in claim 1, is further comprised of a GPS locator device housed inside the Tourniquet.
